(19) United States
(12) Patent Application Publication
(10) Pub. No.: US 2025/0214871 A1
(43) Pub. Date: Jul. 3, 2025

(54) SYSTEM FOR PREDICTING WATER QUALITY IN WATER TREATMENT SYSTEM, AND METHOD FOR PREDICTING WATER QUALITY

(71) Applicant: ORGANO CORPORATION, Tokyo (JP)

(72) Inventors: Yuki NAKAMURA, Tokyo (JP); Yusuke TAKAHASHI, Tokyo (JP); Naoya YOSHINAGA, Tokyo (JP); Kazushige TAKAHASHI, Tokyo (JP)

(73) Assignee: ORGANO CORPORATION, Tokyo (JP)

(21) Appl. No.: 18/848,260

(22) PCT Filed: Feb. 20, 2023
(86) PCT No.: PCT/JP2023/006017

§ 371 Date: Sep. 18, 2024

(30) Foreign Application Priority Data
Mar. 31, 2022 (JP) 2022-060359

(51) Int. Cl. C02F 1/44; C02F 1/32; C02F 1/42; G01N 33/18

(52) U.S. Cl. CPC C02F 1/441; C02F 1/32; C02F 1/42; G01N 33/18; C02F 2209/20

(57) ABSTRACT

A water quality prediction system for predicting quality of treated water in a water treatment system equipped with a first water treatment device that performs a unit operation on water to be treated when the water to be treated is supplied to the water treatment system and the water treatment system is operated based on first operating parameters includes: a system for evaluation that is equipped with a second water treatment that performs same unit operation as the first water treatment device, that is supplied with the water to be treated, and that is operated according to second operating parameters; and a calculation unit that calculate a predicted value of solute concentration in the treated water of the water treatment system based on quality of the water to be treated, quality of treated water in the system for evaluation, the first operating parameters, and the second operating parameters.

### TECHNICAL FIELD

The present invention relates to a system and method for predicting the quality of treated water obtained by a water treatment system.

### BACKGROUND ART

Pure water and ultrapure water are used for cleaning purposes in fields such as fabrication of semiconductor devices. When producing pure water or ultrapure water from raw water, ionic impurities or organic impurities (TOC (Total Organic Carbon) components) contained in the raw water are removed from the raw water in a pure water production system or ultrapure water production system which consists of an ion exchange device, reverse osmosis membrane device, ultraviolet irradiation device, etc. In the following description, the term “pure water” includes ultrapure water, and “pure water production system” includes an ultrapure water production system.

So far, river water, well water, surface water and the like have been used as raw water for pure water production. However, in response to the recent trend toward depletion of water resources, the number of cases using recovered water, which is obtained by treating industrial wastewater and domestic wastewater, as raw water has been increasing. It is known that the concentration, composition and ratio of each component such as ions and TOC in recovered water are significantly different from those in river water or the like. For example, the recovered water may contain persistent TOC components. Persistent TOC components are organic components that are difficult to remove by reverse osmosis membrane treatment, ion exchange treatment, ultraviolet oxidation treatment by ultraviolet irradiation, or the like. If raw water contains persistent TOC, when pure water is produced from the raw water using an existing pure water production systems, a decrease in quality of the resulting pure water may occur. Specifically, an increase in TOC concentration in the resulting pure water may occur. It is required to change the acceptance or rejection of raw water and the operating conditions of the pure water production system depending on water quality of the raw water. In the case of a pure water production system with a large processing capacity, it takes time for the influence of the change in water quality of the supplied raw water to reach the outlet, so it is not appropriate to respond to the change in water quality of the raw water after detecting the change in water quality in the treated water obtained from the outlet. For this reason, it has been proposed to install a small pure water production system, or a system for evaluation, which evaluates the quality of raw water, separately from the pure water production system (main pure water production system) that produces pure water to be supplied to a point-of-use, and to evaluate the quality of raw water by measuring the quality of pure water produced in the system for evaluation.

Patent Literature 1 discloses a water treatment management device used for managing operation of water treatment systems, such as an ultrapure water production system that supplies ultrapure water to a point-of-use. In the technique described in Patent Literature 1, considering the water to be supplied to the water treatment system as target water, an evaluation pure water production unit equipped with TOC removal devices that performs unit operations used to remove TOC components is provided separately from the water treatment system, and the TOC concentrations at multiple measurement points in the evaluation pure water production unit are measured, and then these TOC concentration values are analyzed to evaluate the target water. In the technique described in Patent Literature 1, the supply of raw water to the water treatment system can be controlled according to the evaluation results. For example, if the target water, which is raw water, is evaluated to contain persistent TOC components, the control can be performed, such as not supplying the raw water to the water treatment system.

Patent Literature 2 discloses that when a main ultrapure water production system is installed to produce ultrapure water to be supplied to a point-of-use from raw water, a sub ultrapure water production system is installed to monitor the quality of the raw water to perform controlling. The sub ultrapure water production system has an equivalent configuration to the main ultrapure water production system to produce ultrapure water of similar quality. TOC concentration of the ultrapure water obtained from the sub ultrapure water production system is measured, the quality of the raw water is evaluated based on the TOC concentration, and, based on the evaluation results, the amount or the like of the raw water supplied to the main ultrapure water production system is controlled. In the system described in Patent Literature 2, for example, when the TOC concentration in the ultrapure water obtained from the sub ultrapure water production system is high, the supply of the raw water to the main ultrapure water production system may be stopped, the raw water may be supplied to the main ultrapure water production system via an urea removal device, or the amount of ultraviolet irradiation at an ultraviolet irradiation device may be increased.

Patent Literature 3, which relates to a reverse osmosis membrane device used for desalination of seawater rather than removal of TOC components, discloses accurate prediction of transport parameters of reverse osmosis membranes and operating conditions of the reverse osmosis membrane device by considering concentration polarization phenomena. Similarly, Patent Document 4 discloses predicting the concentration of a specific component in permeated water of a reverse osmosis membrane from the total salt concentration in the permeated water, and setting or controlling the operating conditions of the reverse osmosis membrane device according to the predicted value.

### CITATION LIST

### Patent Literature

Patent Literature 1: JP 2019-155275 A
Patent Literature 2: JP 2016-107249 A
Patent Literature 3: JP 2001-62255 A
Patent Literature 4: JP 2001-129365 A

### SUMMARY OF INVENTION

### Technical Problem

The system for evaluation used to assess the quality of raw water must be able to quickly and conveniently evaluate the impact of the raw water on the main pure water production system with a small amount of the raw water. Therefore, the system for evaluation must be configured to be as small as possible compared to the main pure water production system. However, by employing the small size configuration, the specifications and operating conditions of the system for evaluation will inevitably differ from those of the main pure water production system. For example, with respect to reverse osmosis membrane devices, the main pure water production system combines dozens of 8-inch (20 cm) reverse osmosis (RO) spiral elements and employs operating conditions with high recovery rates of 80 to 95%. In contrast, the system for evaluation system preferably uses one or two small membrane elements, such as 2 to 4 inches (5 to 10 cm). In addition, since small membrane elements that use the same brand of reverse osmosis membrane as that used in the main pure water production system may not always be available, it may be necessary to select a reverse osmosis membrane with different performance than that in the main pure water production system. The recovery rate and flux in the reverse osmosis membrane device are also limited due to the limitations in the amount of raw water that can be used for evaluation and the amount of water supplied to the equipment provided at a subsequent stage of the system for evaluation.

The same is true for an ultraviolet irradiation device for ultraviolet oxidation treatment. Since a small ultraviolet irradiation device with a ultraviolet lamp having the same performance as the ultraviolet lamp used in the main pure water production system may not be available, it may be necessary to choose a small ultraviolet irradiation device with different performance than that in the main pure water production system. In the main pure water production system, membrane degassing devices or oxidizing agent addition devices are sometimes installed to improve the TOC removal rate in the ultraviolet oxidation process. However, installing these devices in the system for evaluation is not necessarily appropriate because installing these devices leads to a large and complicated system. To begin with, the water quality supplied from the reverse osmosis membrane device to the ultraviolet irradiation device differs between the main pure water production system and the system for evaluation, and this difference in water quality has a significant impact on the quality of the treated water from the ultraviolet oxidation treatment.

Thus, existing systems for evaluation can estimate the behavior of TOC components in the treated water (i.e., pure water) obtained from the main pure water production system, but cannot infer detailed values of TOC concentrations in the treated water. The techniques described in the Patent Literatures 1 and 2 do not consider estimating the TOC concentration of the pure water obtained from the main pure water production system.

The above describes the issues related to predicting water quality in pure water production systems. This issue does not occur only in predicting water quality in pure water production systems. A similar issue arises when there is a water treatment system that treats water to be treated in some way to obtain treated water, and when a system for evaluation corresponding to the water treatment system is provided to measure the water quality in the system for evaluation and the water quality of the water to be treated that is supplied to the water treatment system is evaluated based on these measurement results.

The object of the present invention is to provide a system and method for predicting the quality of treated water obtained by a targeted water treatment system using a system for evaluation, which is a smaller water treatment system.

### Solution to Problem

According to an aspect of the invention, the water quality prediction system is a water quality prediction system for predicting quality of treated water in a water treatment system equipped with a first water treatment device that performs a unit operation on water to be treated when the water to be treated is supplied to the water treatment system and the water treatment system is operated based on first operating parameters, the water quality prediction system comprising: a system for evaluation that is equipped with a second water treatment that performs the same unit operation as the first water treatment device, that is supplied with the water to be treated which is to be supplied to the water treatment system, and that is operated according to second operating parameters; and a calculation means that calculate a predicted value of solute concentration in the treated water of the water treatment system based on quality of the water to be treated, quality of treated water in the system for evaluation, the first operating parameters, and the second operating parameters.

According to an aspect of the invention, the water quality prediction method is a method for predicting quality of treated water in a water treatment system equipped with a first water treatment device that performs a unit operation on water to be treated water when the water to be treated is supplied to the water treatment system and the water treatment system is operated based on first operating parameters, comprising: supplying the water to be treated, which is to be supplied to the water treatment system, to a system for evaluation to operate the system for evaluation based on second operating parameters, the system for evaluation being equipped with a second water treatment device that performs the same unit operation as the first water treatment device; and calculating a predicted value of solute concentration of the treated water in the water treatment system based on quality of the water to be treated, quality of treated water in the system for evaluation, the first operating parameters, and the second operating parameters.

In the above water quality prediction system and method, “second water treatment device that performs the same unit operation as the first water treatment device” means that the type of devices constituting the first water treatment device, which is the target system, is the same as the type of devices constituting the second water treatment device which is the system for evaluation. If the first water treatment device is equipped with, for example, a reverse osmosis membrane device, an ultraviolet irradiation device, and an ion exchange device arranged in this order as devices performing unit operations, then the second water treatment device is also equipped with a reverse osmosis membrane device, an ultraviolet irradiation unit, and an ion exchange device arranged unit in this order even if the individual models and specifications of the devices may differ. The solute whose concentration is to be predicted in the present invention is, for example, a TOC component, but substances other than TOC component, such as boron and various ions, can also be used as the solute to be predicted. If TOC is a target of evaluation as solute, then the solute permeation coefficient defined in the reverse osmosis membrane is the TOC permeation coefficient.

The above water quality prediction system and water quality prediction method make it possible to accurately predict the quality of treated water obtained from a target water treatment system using a system for evaluation which a smaller water treatment system.

### BRIEF DESCRIPTION OF DRAWINGS

FIG. 1 is a view showing an example of the overall configuration including a water quality prediction system and a pure water production system which is a target of the water quality prediction;

FIG. 2 is a view illustrating the water quality prediction according to the first embodiment;

FIG. 3 is a view illustrating the water quality prediction according to the second embodiment;

FIG. 4 is a view illustrating the water quality prediction according to the third embodiment;

FIG. 5 is a view illustrating the water quality prediction according to the fourth embodiment;

FIG. 6A is a view illustrating the water quality prediction in Calculation Examples 1 and 2;

FIG. 6B is a view illustrating the water quality prediction in Calculation Examples 1 and 2;

FIG. 6C is a view illustrating the water quality prediction in Calculation Examples 1 and 2;

FIG. 7A is a view illustrating the water quality prediction in Calculation Example 3;

FIG. 7B is a view illustrating the water quality prediction in Calculation Example 3;

FIG. 7C is a view illustrating the water quality prediction in Calculation Example 3;

FIG. 8A is a view illustrating the water quality prediction in Calculation Example 4;

FIG. 8B is a view illustrating the water quality prediction in Calculation Example 4;

FIG. 8C is a view illustrating the water quality prediction in Calculation Example 4; and

FIG. 8D is a view illustrating the water quality prediction in Calculation Example 4.

### DESCRIPTION OF EMBODIMENTS

Next, the embodiments for implementing the present invention will be described with reference to the drawings. In the following, a water treatment system to which water to be treated is supplied to produce treated water and which is the subject of quality prediction of the treated water is referred to as a target system.

The method for predicting water quality according to the present invention is a method using a system for evaluation, which is configured as a water treatment system similar to the target system but smaller than the target system, when the target system is available, and predicting, based on the measurement results of water quality in the system for evaluation, a detailed value of the quality of the treated water produced by the target system. More specifically, in this water quality prediction method, water to be treated is supplied to a target system equipped with a first treatment device that performs a unit operation on the water to be treated, and the water to be treated is also supplied to a system for evaluation equipped with a second treatment device that performs the same type of unit operation as the unit operation performed in the target system in order to predict the water quality in the target system when the target system is operated based on the first operation parameters to obtain the treated water. Then, the system for evaluation is operated based on the second operating parameters, and the solute concentration in the target system is calculated based on the quality of the water to be treated, the water quality in the system for evaluation, the first operating parameters, and the second operating parameters. When the quality of the treated water from the target system is predicted in this way, if the predicted value obtained deviates from the target water quality in the target system, the operating parameters of the target system, i.e., the first operating parameters, can be changed so that the quality of the treated water approaches the target water quality.

The water treatment system, or target system, to which the water quality prediction method based on the present invention is applied is not particularly limited, but in the following description, it is assumed that the water treatment system is a pure water production system that produces pure water from raw water, which is the water to be treated, and supplies the pure water to a point-of-use, for example. The water quality which is target of measurement in the system for evaluation and also target of evaluation in the pure water production system is considered to be the concentration of impurities, which is the solutes, more specifically, the concentration of TOC components dissolved in water, which is the solvent, as solutes. Therefore, the following describes a case in which, when a target pure water production system is available, a water quality prediction system equipped with a system for evaluation configured as a pure water production system smaller than the target pure water production system is used to predict detailed values of TOC concentration of the pure water produced by the target pure water production system based on the measurement results of TOC concentration in the system for evaluation. The term “small” here means that at least one of the devices constituting the system for evaluation is smaller than the corresponding device in the target system. The case that each of the target system and the system for evaluation is equipped with a reverse osmosis membrane device, a ultraviolet irradiation device, and an ion exchange device provided in this order is considered. For example, if the reverse osmosis membrane device of the system for evaluation is smaller than the the reverse osmosis membrane device of the target system, then the system for evaluation corresponds to the smaller system for evaluation than the target system. Of course, all of the devices constituting the system for evaluation may be smaller than the corresponding devices in the target system. In the examples given here, the devices in the system for evaluation may be smaller than the corresponding devices in the target system for all of the reverse osmosis membrane devices, ultraviolet irradiation devices, and ion exchange devices.

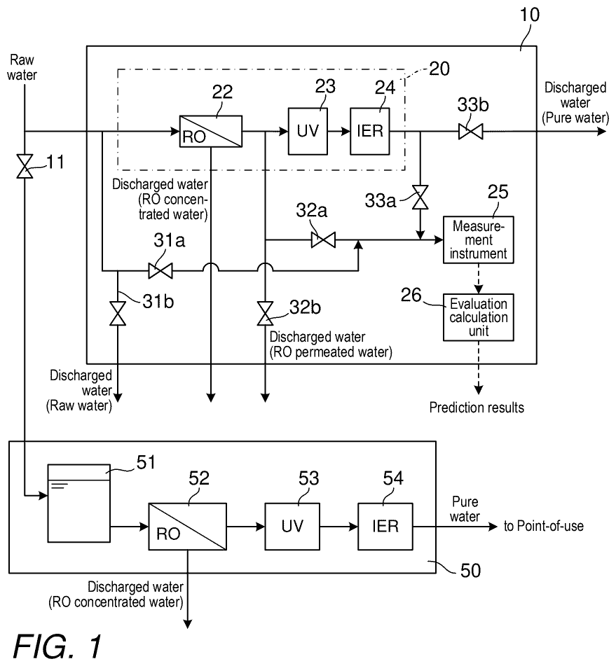

FIG. 1 is a view for illustrating the water quality prediction method and shows an example of the overall configuration, including water quality prediction system 10 and pure water production system 50 which is the water treatment system to be evaluated. In each of the following embodiments, the water quality prediction method is applied to the overall configuration shown in FIG. 1. Raw water containing unknown TOC components is supplied to water quality prediction system 10 and also to pure water production system 50 to be evaluated via valve 11. Pure water production system 50 is a large system configured to provide pure water to s point-of-use. Pure water production system 50 is equipped with: tank 51 that temporarily stores raw water; reverse osmosis membrane device (RO) 52 to which the raw water in tank 51 is supplied; ultraviolet irradiation device (UV) 53 to which the permeated water (RO permeated water) from reverse osmosis membrane device 52 is supplied to perform ultraviolet oxidation treatment; and ion exchange device (IER) 54 that performs ion exchange treatment to the treated water from ultraviolet irradiation device 53. In this pure water production system 50, the treated water from ion exchange device 54 is supplied to the point-of-use as pure water. Water (RO concentrated water) that does not permeate the reverse osmosis membrane in reverse osmosis membrane device 52 is discharged outside as it is.

In pure water production system 50, a membrane degassing device that performs degassing treatment on the RO permeated water, and a device that adds an oxidizing agent such as hydrogen peroxide may be provided in the preceding stage of ultraviolet irradiation device 53 to increase the TOC removal rate in the ultraviolet oxidation treatment, but these devices are not shown in FIG. 1. The raw water described here may be water which have been treated in advance by equipment associated with pure water production system 50. For example, water that has undergone pretreatment, such as a sand filtration device, activated carbon treatment device, ion exchange device, and degassing device which are associated with pure water production system 50 may be branched and fed to system 10 for evaluation.

Water quality prediction system 10 is equipped with system 20 for evaluation in which the raw water is supplied to produce pure water from the raw water for evaluating the raw water. System 20 for evaluation is equipped with: reverse osmosis membrane device (RO) 22 to which the raw water is supplied; ultraviolet irradiation device (UV) 23 which is supplied with the permeated water (RO permeated water) from reverse osmosis membrane device 22, and performs ultraviolet oxidation treatment on this water; and ion exchange device (IER) 24 which is supplied with the outlet water from the ultraviolet irradiation device 23 and performs ion exchange treatment. The outlet water from ion exchange device 24 is the treated water from system 20 for evaluation. In addition, water quality prediction system 10 is equipped with measurement instrument 25 which measures TOC concentration. A portion of the raw water supplied to system 20 for evaluation is branched and supplied to measurement instrument 25 via valve 31a, a portion of the RO permeated water is branched and supplied to measurement instrument 25 via valve 32a, and a portion of the outlet water of ion exchange device 24 is supplied to measurement instrument 25 via valve 33a. In water quality prediction system 10, by controlling the opening and closing of valves 31a to 33a, the raw water, RO permeated water, and treated water after ion exchange treatment can be switched to measure the TOC concentration of those waters. Water quality prediction system 10 includes evaluation calculation unit 36 that predicts the TOC concentration in the pure water produced from the raw water by pure water production system 50 and the TOC concentration at each point in pure water production system 50, based on the measurement results at measurement instrument 25 and the operating parameters described below. From the perspective of protecting water quality prediction system 10 and improving prediction accuracy, the raw water may be passed through a pretreatment device such as a heat exchanger, filter, activated carbon treatment device, ion exchange device, or degassing device, and the raw water treated by the pretreatment device may be accepted into water quality prediction system 10.

In the configuration shown in FIG. 1, system 20 for evaluation in water quality prediction system 10 and pure water production system 50 to be evaluated are the same in that the raw water is passed through reverse osmosis membrane devices 22, 52, ultraviolet irradiation devices 23, 53 and ion exchange devices 24, 54 in this order and treated to produce pure water. The major difference between system 20 for evaluation and pure water production system 50 is that pure water production system 50 is a large-scale system used to supply a large amount of pure water to point-of use, whereas system 20 for evaluation is a small-scale system equipped with measurement instrument 25 and evaluation calculation unit 26, and is used to predict water quality (especially TOC concentration) in pure water production system 50.

### First Embodiment

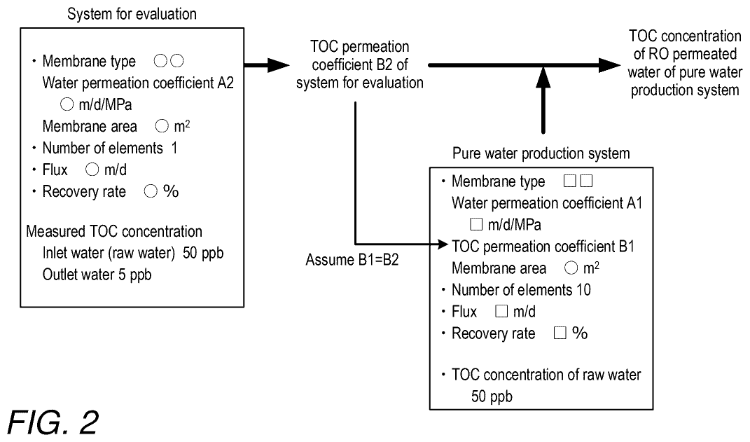

Next, the water quality prediction in the configuration shown in FIG. 1 will be explained. The ultimate goal of the water quality prediction is to quickly predict the water quality (especially TOC concentration) of pure water produced by pure water production system 50, which are large and slow to respond to changes in quality of the raw water, based on measurement results taken by system 20 for evaluation. In both system 20 for evaluation and pure water production system 50, the raw water is passed through reverse osmosis membrane device 22, 52, ultraviolet irradiation device 23, 53, and ion exchange device 24, 54 in this order, thereby removing TOC components from the raw water. In reverse osmosis membrane devices 22, 52, TOC components with large molecular weight or electrical charging properties are removed. The remaining TOC components are converted into components such as organic acids and carbonic acids by the ultraviolet oxidation treatment in ultraviolet irradiation devices 23, 53, and then the components such as organic acids and carbonic acids are removed together with other remaining ionic impurities in ion exchange devices 24, 54. In terms of removal of TOC components, the series of treatments can be broadly divided into the treatment in reverse osmosis membrane devices 22, 52, and the treatment in ultraviolet irradiation devices 23, 53 and ion exchange devices 24, 54. The TOC concentration in the pure water finally obtained by pure water production system 50 depends on how much of the TOC components are removed in each of these processes. Therefore, the first embodiment describes an example of predicting the TOC concentration of the treated water, or RO permeated water, in reverse osmosis membrane device 52 of pure water production system 50, from the quality of the treated water, or RO permeated water, in reverse osmosis membrane device 22 of system 20 for evaluation. FIG. 2 illustrates the water quality prediction in the first embodiment.

The configuration of reverse osmosis membrane device 22 in system 20 for evaluation and the type (membrane type) of the reverse osmosis membrane used therein are known, and the operating conditions thereof are also known. The configuration of reverse osmosis membrane device 22 includes the membrane area, number of membrane elements, etc. The type of reverse osmosis membrane is also referred to as membrane type. The operating conditions include recovery rate and flux in reverse osmosis membrane device 22. Here, flux refers to the permeation flux in a reverse osmosis membrane. If the membrane type is known, water permeation coefficient A2, which is a solvent permeation coefficient, intrinsic to the reverse osmosis membrane, is also known. The water permeation coefficient is expressed in units of m/d/MPa. Similarly, the configuration and membrane type of reverse osmosis membrane device 52 in pure water production system 50 are known, the operating conditions thereof are known, and water permeation coefficient A1 in the reverse osmosis membrane is known. Naturally, the operating conditions can be set as desired. In system 20 for evaluation, the TOC concentration of the inlet water, i.e., the raw water, of reverse osmosis membrane device 22 and the TOC concentration of the RO permeated water are also known by measurement with measurement instrument 25. On the other hand, in pure water production system 50, since its reverse osmosis membrane device 52 is supplied with the same raw water as system 20 for evaluation, the TOC concentration of the inlet water of reverse osmosis membrane device 52 is known. What we want to find in the present embodiment is the TOC concentration in the RO permeated water (i.e., the treated water) from reverse osmosis membrane device 52 in pure water production system 50. This TOC concentration is a TOC concentration derived from the unknown TOC components contained in the raw water. The membrane type, membrane area, number of membrane elements, operating conditions, water permeation coefficient, and so on are collectively referred to as the operating parameters of a reverse osmosis membrane device.

First, TOC permeation coefficient B2, which is the permeation coefficient (solute permeation coefficient) of the TOC component in the reverse osmosis membrane of reverse osmosis membrane device 22 in system 20 for evaluation is determined. The TOC permeation coefficient, which is a solute permeation coefficient, is expressed in units of m/d. TOC permeation coefficient B2 can be calculated from the TOC concentration at both sides of the reverse osmosis membrane, i.e., TOC concentrations in the inlet water and RO permeated water, and the flux, by membrane transport parameter calculation using a concentration polarization model, as described, for example, in Patent Literatures 3 and 4. Next, assuming that TOC permeation coefficient B2 in reverse osmosis membrane device 22 in system 20 for evaluation and TOC permeation coefficient B1 in reverse osmosis membrane device 52 in pure water production system 50 are the same, the TOC concentration of the RO permeated water of reverse osmosis membrane device 52 of pure water production system 50 is calculated from the TOC concentration of the inlet water in pure water production system 50, the membrane area, recovery rate, and flux. If the reverse osmosis membrane used in the pure water production system and the reverse osmosis membrane used in system 20 for evaluation are of equivalent performance as membranes, it is a reasonable assumption that TOC permeation coefficients B1 and B2 are the same for the unknown TOC component to be predicted. The TOC concentration in the RO permeated water of reverse osmosis membrane device 52 in pure water production system 50 can be predicted from the measurement results in system 20 for evaluation. In this embodiment, parameters due to the mechanical structure in reverse osmosis membrane devices 22, 52 may be further considered.

In the water quality prediction system shown in FIG. 1, the operating parameters of reverse osmosis membrane device 22 of system 20 for evaluation and the operating parameters of pure water production system 50 are set in advance in evaluation calculation unit 26. Upon receiving the measured values of the TOC concentration of the raw water and RO permeated water of reverse osmosis membrane device 22 from measurement instrument 25, evaluation calculation unit 26 calculates and outputs the predicted value of the TOC concentration in the RO permeated water of reverse osmosis membrane device 52 in pure water production system 50 as described above, and outputs the predicted value.

### Second Embodiment

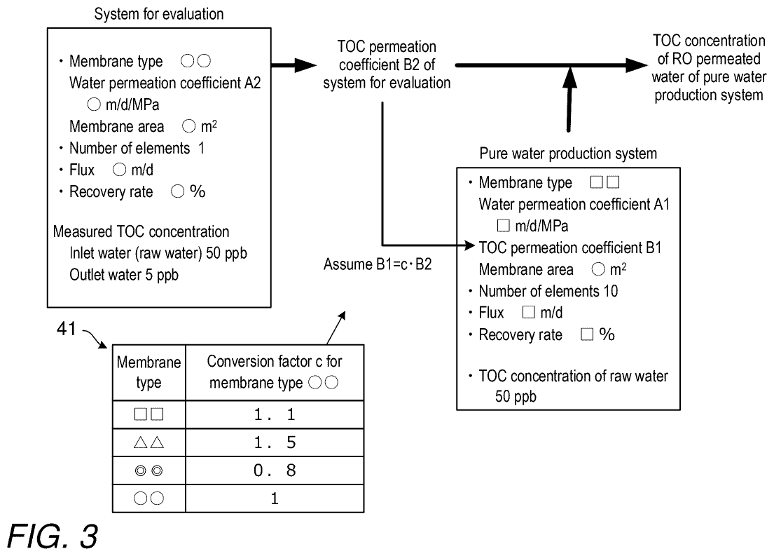

The water quality prediction method according to the second embodiment will be described using FIG. 3. If reverse osmosis membrane device 52 in pure water production system 50 and reverse osmosis membrane device 22 in system 20 for evaluation are different in membrane type, especially if the physical or chemical properties of the reverse osmosis membrane are significantly different, the assumption that TOC permeation coefficient B1 in reverse osmosis membrane device 52 is the same as TOC permeation coefficient B2 in reverse osmosis membrane device 22 is no longer valid. FIG. 3 illustrates the water quality prediction method for such cases. When different membrane types are used, conversion factors c for the membrane type used in system 20 for evaluation 20 are determined for each membrane type that may be used in pure water production system 50, and these conversion factors c are stored in advance in the form of conversion table 41 (see FIG. 3) in a database provided in evaluation calculation unit 26. Once TOC permeation coefficient B2 in system 20 for evaluation system 20 is determined, TOC concentration in the RO permeated water of reverse osmosis membrane device 52 in pure water production system 50 can be predicted by determining TOC permeation coefficient B1 in pure water production system 50 by calculating B1=c·B2, and then performing the same procedure as in the first embodiment. In this embodiment, when the combination of the membrane type used in system 20 for evaluation and the membrane type used in pure water production system 50 are input, evaluation calculation unit 26 searches built-in conversion table 41 to read out the corresponding conversion factor c and uses conversion factor c to perform the prediction calculation of the TOC concentration of the RO permeated water in pure water production system 50. Since the first embodiment described above is equivalent to the case where c=1 in the second embodiment, if c=1 is specified in conversion table 41 for the case where the same membrane type is used in system 20 for evaluation and pure water production system 50, it is possible to predict water quality based on the second embodiment in a form that includes the first embodiment.

Conversion factor c for each membrane type in the second embodiment can be determined base on measurement results obtained by, for example, measuring the TOC permeation coefficient for each membrane type using known TOC components. Known TOC components for determining the TOC permeation coefficient can be low molecular weight organic substances with a molecular weight of about 100 or less, such as isopropyl alcohol, urea, and ethanol. Instead of these low-molecular-weight organic substances, boric compounds such as boric acid can be used, although not strictly organic.

### Third Embodiment

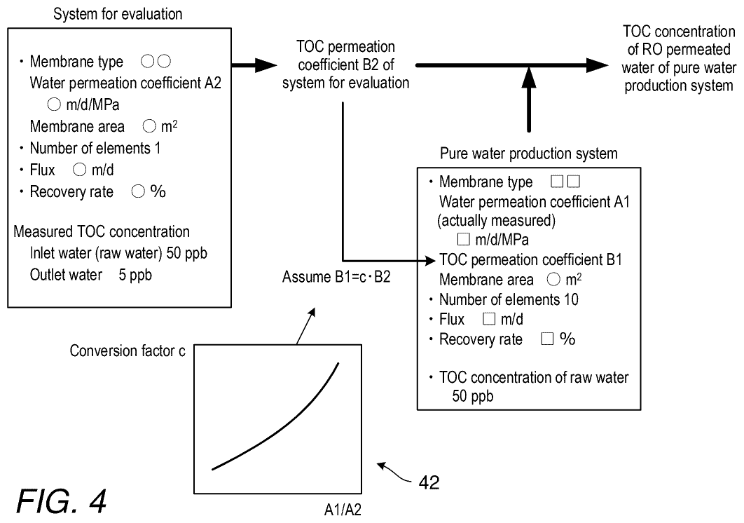

In the second embodiment, conversion factor c is determined for each membrane type, or more precisely, for each combination of the membrane type used in pure water production system 50 and the membrane type used in system 20 for evaluation, and TOC permeation coefficient B2 obtained in reverse osmosis membrane device 22 of system 20 for evaluation is linked to TOC permeation coefficient B1 in reverse osmosis membrane device 52 of pure water production system 50 via conversion factor c. However, when the performance of a reverse osmosis membrane has changed from its initial performance, for example, when the reverse osmosis membrane deteriorates or clogs, it may become inappropriate to apply conversion factor c that is predetermined for each membrane type. If the membrane type of the reverse osmosis membrane used in pure water production system 50 is unknown, it is impossible to know conversion factor c in the first place. Here, cases where the performance of a reverse osmosis membrane has changed from its initial performance are also included when the membrane type is unknown. In the third embodiment, the TOC concentration of the RO permeated water is predicted in cases where it is inappropriate or unavailable to use existing conversion factor c. FIG. 4 illustrates the processing in the third embodiment. The third embodiment does not differ from the second embodiment in that TOC removal rate B2 in reverse osmosis membrane device 22 in system 20 for evaluation multiplied by conversion factor c is used as TOC removal rate B1 in reverse osmosis membrane device 52 in pure water production system 50. The third embodiment differs from the second embodiment in that it estimates the value of conversion factor c.

In the third embodiment, water permeation coefficient A1 in reverse osmosis membrane device 52 of pure water production system 50 is first calculated. If the membrane type is unknown, or if the reverse osmosis membrane is deteriorating or blocked, water permeation coefficient A1 itself will change. It is necessary to calculate water permeation coefficient A1. Water permeation coefficient A1 can be determined using the conductivity, pressure, and the like in each of the inlet water, RO permeate water, and RO concentrate water, in addition to the operating parameters, such as flux, in reverse osmosis membrane device 52. Once water permeation coefficient A1 is calculated, the ratio (A1/A2) of this water permeation coefficient A1 to water permeation coefficient A2 in system for evaluation 20 is determined, conversion factor c is determined based on the ratio (A1/A2), and the TOC concentration of the RO permeated water in pure water production system 50 is calculated using the determined conversion factor c. According to the findings by the inventors, when there are two types of reverse osmosis membranes, there is a correlation between the ratio (A1/A2) of the water permeability coefficients between those membranes and the ratio (B1/B2) of the TOC permeability coefficients, or conversion factor c. Therefore, this correlation is determined in advance and stored in a database in evaluation calculation unit 26 of water quality prediction system 10. In the third embodiment, water permeation coefficient A1 actually measured with respect to reverse osmosis membrane device 52 of pure water production system 50 is input to evaluation calculation unit 26 instead of the membrane type. Evaluation calculation unit 26 then calculates the ratio (A1/A2) of the above-mentioned water permeation coefficients, applies this ratio (A1/A2) to the above-mentioned correlation stored in advance to obtain conversion coefficient c, and then calculates the TOC concentration of the RO permeated water in pure water production system 50 in the same way as in the second embodiment.

In FIG. 4, graph 62 shows an example of the correlation between the ratio (A1/A2) of water permeation coefficients and conversion factor c. This correlation is determined by prior experimentation. For example, water containing an indicator substance as a TOC component can be passed through reverse osmosis membranes 22 in system 20 for evaluation 20 and multiple reverse osmosis membrane elements each equipped with a reverse osmosis membrane with a TOC permeation coefficient different from that in system 20 for evaluation to determine the TOC permeation coefficients, so that the correlation can be determined. Low molecular weight organic substances with a molecular weight of about 100 or less can be used preferably as indicator substances.

### Fourth Embodiment

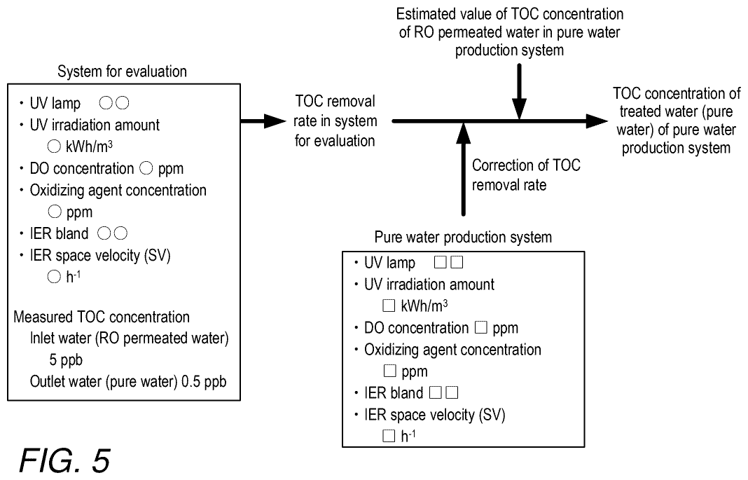

The first to third embodiments predict the TOC concentration in the RO permeated water discharged from reverse osmosis membrane device 52 of pure water production system 50. To predict the TOC concentration in the final pure water obtained from pure water production system 50, it is necessary to estimate the TOC removal rate in the ultraviolet oxidation treatment and the subsequent ion exchange treatment to which the RO treated water is fed, and apply the TOC removal rate to the TOC concentration in the RO permeated water to calculate the final TOC concentration. The fourth embodiment relates to an integrated treatment of the ultraviolet oxidation treatment and the ion exchange treatment, i.e., ultraviolet oxidation-ion exchange treatment, and prediction of the TOC removal rate thereof. FIG. 5 illustrates the fourth embodiment.

For the ultraviolet oxidation-ion exchange treatment, the operating parameters of system 20 for evaluation include: the type and specifications of the ultraviolet (UV) lamps used in ultraviolet irradiation device 23; ultraviolet irradiation amount; dissolved oxygen (DO) concentration in the inlet water; oxidizing agent concentration in the inlet water after adding an oxidizing agent when the oxidizing agent such as hydrogen peroxide is added to the inlet water; brand of the ion exchange resin (IER) used in ion exchange device 24; space velocity (SV) of the water flow in the ion exchange resin; and so on. Items other than those listed here may be included in the operating parameters. For example, the concentration of dissolved carbon dioxide (CO₂) in the inlet water may be included in the operating parameters. If multiple ion exchange resins are mixed and used in ion exchange device 24, their mixing ratio is also included in the operating parameters. The inlet water here is the RO permeated water from reverse osmosis membrane device 22 provided the a preceding stage, which is supplied to ultraviolet irradiation device 23. The outlet water from the ultraviolet oxidation-ion exchange treatment is the treated water (pure water) from ion exchange device 24. In the fourth embodiment, the TOC removal rate for unknown TOC components derived from the raw water in the ultraviolet oxidation-ion exchange treatment is first calculated based on the TOC concentration in the inlet water and the TOC concentration in the outlet water of the ultraviolet oxidation-ion exchange treatment in system 20 for evaluation.

By implementing the methods described in the first to third embodiments, the predicted value of the TOC concentration in the RO permeated water of reverse osmosis membrane device 52 in pure water production system 50 have been obtained. The TOC concentration of the treated water (pure water) from the ultraviolet oxidation-ion exchange treatment in pure water production system 50 is then predicted by applying the TOC removal rate in the ultraviolet oxidation-ion exchange treatment obtained in system 20 for evaluation to the predicted value of the TOC concentration of the RO permeated water. At this time, taking into account the configuration differences between system 20 for evaluation and pure water production system 50, the TOC removal rate to be used is corrected, for example, as shown in (1) to (4) below.
(1) For example, if the ultraviolet irradiation efficiency differs depending on the manufacturer and part number of the ultraviolet lamp, a correction factor for the irradiation efficiency is obtained in advance for each lamp and multiplied to the TOC removal rate.
(2) In general, the removal efficiency of TOC in ultraviolet oxidation-ion exchange treatment tends to be lower the higher the TOC concentration in the inlet water. Therefore, if the predicted TOC concentration for the RO permeated water in pure water production system 50 is higher than the measured TOC concentration for the RO permeated water in system 20 for evaluation, the TOC removal rate is adjusted downward.
(3) It is also known that the removal efficiency of TOC by ultraviolet oxidation-ion exchange treatment varies depending on the dissolved oxygen concentration. Therefore, correction factors between the dissolved oxygen concentration and the removal efficiency are obtained in advance, and if the dissolved oxygen concentration differs between system 20 for evaluation and pure water production system 50, the correction factor corresponding to the difference in dissolved oxygen concentration are multiplied to the TOC removal rate.
(4) It is also known that the removal efficiency of TOC by ultraviolet oxidation-ion exchange treatment varies depending on the concentration of oxidizing agents. Therefore, correction factors between the oxidizing agent concentration and the removal efficiency are obtained in advance, and if the oxidizing agent concentration differs between system 20 for evaluation 20 and pure water production system 50, the correction factor corresponding to the difference in oxidizing agent concentration is multiplied to the TOC removal rate.

In water quality prediction system 10, evaluation calculation unit 26 calculates the TOC removal rate of the ultraviolet oxidation-ion exchange treatment in system 20 for evaluation based on the measurement results from measurement instrument 25, and calculates the predicted value of the TOC concentration in the RO permeated water of reverse osmosis membrane device 52 of pure water production system 50 based on the method described in the first to third embodiments. Evaluation calculation unit 26 then calculates the predicted value of the TOC concentration in the treated water (pure water) of pure water production system 50 by correcting the TOC removal rate, which has been calculated in this way, as described above and then applying the corrected TOC removal rate to the predicted value of the TOC concentration in the RO permeated water of pure water production system 50. If the quality of the inlet water of ultraviolet UV irradiation device 23 in system 20 for evaluation and the inlet water of ultraviolet irradiation device 53 in pure water production system 50 are considered to be equivalent, including cases where reverse osmosis membrane units 22, 52 are not installed, the correction for the RO permeated water as shown in the first to third embodiments does not need to be performed. Instead of using the predicted value of the TOC concentration for the RO permeated water of reverse osmosis membrane device 52 of pure water production system 50, the treated water quality of ion exchange device 54 of pure water production system 50 may be calculated from the quality of the treated water of ion exchange device 24 of system 20 for evaluation and the first and second operating parameters.

In the above description, an ion exchange device filled with ion exchange resin (IER) is used for the ion exchange treatment, but an electrodeionization (EDI) device may be used instead of an ion exchange device in both system 20 for evaluation and pure water production system 50. In an ordinary ion exchange device, the water quality may decrease due to breakage or the like of the ion exchange resin after prolonged use, and it is necessary to replace the ion exchange device or regenerate the ion exchange resin, but in an EDI device, since the ion exchange treatment and regeneration treatment of the ion exchanger occur simultaneously, so there is no risk of water quality degradation. In the above explanation, it has been stated that a membrane degassing device or the like may also be provided in pure water production system 50, in addition to reverse osmosis membrane device 52, ultraviolet irradiation device 53, and ion exchange device 54. Also in system 20 for evaluation, a membrane degassing device or EDI device may be placed in a preceding stage of ultraviolet irradiation device 23 to reduce the dissolved oxygen and dissolved carbon dioxide concentrations in the inlet water of the ultraviolet oxidation treatment. By keeping the dissolved oxygen and dissolved carbon dioxide concentrations in the inlet water of the ultraviolet oxidation treatment sufficiently low, accuracy can be improved when predicting the TOC concentration in the treated water of pure water production system 50.

The water quality prediction system and water quality prediction method based on the present invention can be suitably used for the production of pure water and ultrapure water, and can also be used for wastewater recovery systems that perform deionization treatment and removal treatment of TOC components on various wastewater generated in industrial plants for recovery and reuse as utility water or water for facilities. In the wastewater recovery systems, where water quality fluctuates depending on the type and quantity of wastewater that merges, the application of the present invention will enable early assessment of the fluctuations in the treated water quality. The water treatment system to which the present invention is applied does not necessarily have to be equipped with all of a reverse osmosis membrane device, an ultraviolet irradiation device, and an ion exchange device, but may have only one or some of these devices. It may even be equipped with other devices that perform some treatment (i.e., unit operation) on the water to be treated without any of these devices. Ultimately, the water treatment system to which the present invention is applied need only be equipped with one or more of the following devices: a reverse osmosis membrane device, an ultraviolet irradiation device, an ion exchange device, a degassing device, an activated carbon device, a distillation device, etc.

For example, if pure water production system 50, which is the water treatment system, is equipped with reverse osmosis membrane device 52 but does not have ultraviolet irradiation device 53 and ion exchange device 54, system 20 for evaluation is also equipped with only reverse osmosis membrane device 22. Then, by applying any of the first to third embodiments described above, the TOC concentration in the permeated water (i.e., treated water) of reverse osmosis membrane device 52 of pure water production system 50 can be predicted. If pure water production system 50, which is the water treatment system, is equipped with ultraviolet irradiation device 53 and ion exchange device 54 installed in a subsequent stage thereof, but does not have reverse osmosis membrane device 52, system 20 for evaluation also consists of ultraviolet irradiation device 23 and ion exchange device 24 installed in a subsequent stage thereof. In this case, by treating the water supplied to ultraviolet UV irradiation devices 23, 53 in the fourth embodiment as the raw water, and measuring the water quality in the raw water, the TOC concentration in the outlet water (i.e., treated water) of ion exchange device 54 in pure water production system 50 can be predicted even if reverse osmosis membrane devices 22, 52 are not provided.

In each of the above described embodiments, water quality prediction system 10 is used to predict the quality of the treated water of pure water production system 50 which is a target of evaluation. Such water quality prediction is usually made to maintain the quality of the treated water of pure water production system 50 within a desired range. Therefore, we will explain the operations that should be performed on each operating parameter of pure water production system 50 when the predicted water quality with respect to pure water production system 50 deviates from the target water quality of pure water production system 50. For example, if the predicted value for the TOC concentration of the treated water in reverse osmosis membrane device 52 in pure water production system 50 is higher than the target value, i.e., if the water quality is poor, the recovery rate of this reverse osmosis membrane device 52 can be reduced or the supply water temperature can be reduced, to bring the predicted TOC concentration for the treated water in reverse osmosis membrane device 52 closer to the target value. Conversely, if the predicted TOC concentration of the treated water in reverse osmosis membrane device 52 is lower than the target value, the recovery rate can be increased or the degree of cooling of the supplied water can be reduced, in order to save energy.

Similarly, if the predicted TOC concentration of the treated water in ultraviolet irradiation device 53 of pure water production system 50 is higher than the target value, the predicted TOC concentration of the treated water of ultraviolet irradiation device 53 can be brought closer to the target value by increasing the ultraviolet irradiation amount in ultraviolet irradiation device 53, performing treatment of lowering the dissolved oxygen concentration in the water supplied to ultraviolet irradiation device 53, increasing the concentration of an oxidizing agent added to the supplied water, lowering the recovery rate of reverse osmosis membrane unit 52 provided in the preceding stage, or lowering the temperature of the water supplied to reverse osmosis membrane unit 52 provided in the preceding stage. Conversely, if the predicted TOC concentration of the treated water in ultraviolet irradiation device 53 is lower than the target value, each operating parameter may be changed in the direction of higher TOC concentration in order to save energy.

### EXAMPLES

Next, the present invention will be explained in more detail with actual calculation examples, i.e., examples of predicting the quality of treated water in pure water production system 50 based on measurement values in system 20 for evaluation. In the following explanation, flow rate of the supplied water (inlet water), flow rate of the concentrated water, and flow rate of the permeated water of the reverse osmosis membrane device are denoted by Qf, Qc, and Qp, respectively. Similarly, the concentrations of solutes (in this case, TOC components) in the supplied water, concentrated water, and permeated water of the reverse osmosis membrane device are denoted by Cf, Cc, and Cp, respectively. Let Jv denote the flux of solvent (in this case, water) in the reverse osmosis membrane device, P the solute permeation coefficient, and Lp the solvent permeation coefficient. Assuming that the concentration polarization model is applied to reverse osmosis membranes, as shown in Patent Literature 3 and 4, the solute concentration at the supply side surface of the reverse osmosis membrane is expressed as Cm. The density of the liquid is p, the viscosity coefficient is n, the channel thickness is d, the flow velocity is u, the diffusion coefficient of the solute is D, and the mass transfer coefficient through the membrane is k. Reynolds number Re, Schmidt number Sc, and Sherwood number Sh are respectively represented by

$$Re = ρ \cdot u \cdot d / η ,$$

$$Sc = η / ( ρ \cdot D ) ,$$

$$Sh = k \cdot d / D .$$

### Calculation Example 1

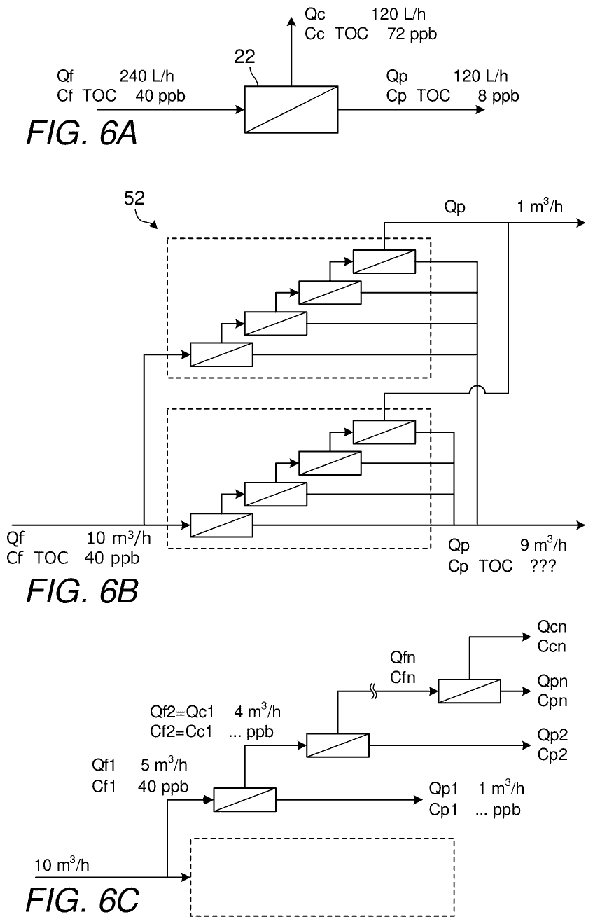

Calculation Example 1 corresponding to the first embodiment is illustrated by using FIGS. 6A to 6C. In Calculation Example 1, pure water was produced by supplying raw water containing an unknown TOC component as a solute to each of system 20 for evaluation and pure water production system 50. FIG. 6A shows flow rates and concentrations in reverse osmosis membrane device 22 of system 20 for evaluation, FIG. 6B shows the configuration of reverse osmosis membrane device 52 of pure water production system 50, and FIG. 6C shows another calculation example of the concentration in reverse osmosis membrane device 52 of pure water production system 50. As reverse osmosis membrane device 22 in system 20 for evaluation, a device equipped with a single 4-inch element ESPA2-4021, manufactured by Nitto Denko Corporation, was used. Its membrane area was 3.5 m². Reverse osmosis membrane device 22 was operated at a recovery rate of 50% and a flux Jv of 0.82 m/d. The flow rate of the supplied water, Qf, to reverse osmosis membrane device 22 was 240 L/h, the flow rate of the concentrated water, Qc, was 120 L/h, and the flow rate of the permeated water, Qp, was also 120 L/h. The solute concentrations of the supplied water, Cf, the concentrated water, and the permeated water in reverse osmosis membrane device 22 were 40 ppb, 72 ppb, and 8 ppb as TOC concentrations, respectively.

First, the solute (TOC) concentration Cm at the membrane surface is calculated. This calculation requires a mass transfer coefficient k, which can be calculated from Sherwood number Sh, channel thickness d, and solute diffusion constant D. For Sherwood number Sh, the Deissler equation is known, using Reynolds number Re and Schmidt number Sc, assuming that α, β, and γ are constants to be obtained by experiment, it can be formulated as

$$Sh = α \times {Re}^{β} \cdot {Sc}^{γ} .$$

In addition, the following stands as described in Patent Literature 3,

$$( Cm - Cp ) / ( Cf - Cp ) = exp ( Jv / k ) . \qquad ( 1 )$$

Since the concentrations Cf, Cc, and Cp are known as described above, the membrane surface concentration of the solute, Cm, can be determined. And, the following stands.

$$Jv \cdot Cp = P ( Cm - Cp ) . \qquad ( 2 )$$

Therefore, when solute permeation coefficient P (i.e., TOC permeation coefficient B2) was determined using equations (1) and (2), P=5.32×10⁻⁷ m/d was obtained.

As reverse osmosis membrane device 52 of pure water production system 50, a device equipped with eight elements, each element being an 8-inch element ES20-D8, manufactured by by Nitto Denko Corporation, was used. In this reverse osmosis membrane device 52, two tracks each of which consists of four membrane elements connected in a cascade are installed in parallel. as shown in FIG. 6B. The membrane area was 37 m². Reverse osmosis membrane device 52 was operated with a recovery rate of 90% and flux Jv of 0.72 m/d. Supplied water flow rate Qf of reverse osmosis membrane device 52 was 10 m³/h, concentrated water flow rate Qc was 1 m³/h, and permeated water flow rate was 9 m³/h. Solute concentration Cf of the supplied water was the same as that in system 20 for evaluation and was 40 ppb as TOC concentration. The membrane performance of the reverse osmosis membrane used in system 20 for evaluation and the reverse osmosis membrane used in pure water production system 50 are almost the same. In this case, solute permeation coefficient P (i.e., TOC permeation coefficient B2) obtained in system 20 for evaluation for the unknown TOC components contained in the raw water can be used as is for the concentration calculation of the aforementioned unknown TOC components in pure water production system 50. Combining equations (1) and (2) above, the following is obtained.

$$Jv \cdot Cp / P = ( Cf - Cp ) \cdot exp ( Jv / k ) \qquad ( 3 )$$

Mass transfer coefficient k was determined as in the case of system 20 for evaluation, and solute concentration Cp was determined from the known values Jv, P, and Cf. Cp was 6.5 ppb. This means that 6.5 ppb was obtained as the predicted TOC concentration of the RO permeated water of reverse osmosis membrane device 52 in pure water production system 50.

The calculation example above is an example in which the calculation was performed by treating the eight membrane elements of reverse osmosis membrane device 52 in pure water production system 50 as a single unit. As shown in FIG. 6C, the above calculation can be performed for each membrane element, and the calculation results can be reflected in the calculation for the membrane element in the subsequent stages. Assuming that the index for the n-th membrane element is n, calculation is first performed for the first-stage membrane element in the manner as described above, then solute concentrations Cp1 and Cc1 in the permeated and concentrated water of the first-stage membrane element are obtained, respectively. Since the concentrated water from the first-stage element is supplied to the second-stage membrane element, the relationship Qc1=Qf2 and Cc1=Cf2 holds. The calculation can be done in this way up to the last stage of membrane elements. From the flow rates of the permeated water and permeated water in each stage, the flow rate and water quality can be calculated when the permeated water from each stage is combined.

### Calculation Example 2

As Calculation Example 2, a calculation example is described for the case of the second embodiment, i.e., the case where the membrane performance differs between the reverse osmosis membrane used in system 20 for evaluation and the reverse osmosis membrane used in pure water production system 50, and the solute permeation coefficient (TOC permeation coefficient) cannot be directly applied. The same system 20 for evaluation as that in Calculation Example 1 was used and operated under the same operating conditions. Therefore, solute permeation coefficient P (i.e., TOC permeation coefficient B2) in system 20 for evaluation with respect to unknown TOC components in the raw water is 5.32×10⁻⁷ m/d. On the other hand, as reverse osmosis membrane device 52 of pure water production system 50, used is a device which is the same as in Calculation Example 1 in that eight membrane elements are connected as shown in FIG. 6B, but different from Calculation Example 1 in that an 8-inch element CPA5-LD, manufactured by Nitto Denko Corporation, is used for each membrane element. The membrane area was 37 m². Reverse osmosis membrane device 52 was operated with a recovery rate of 90% and flux Jv of 0.72 m/d.

If the solute permeation coefficients in pure water production system 50 and system 20 for evaluation are represented by P1 and P2, respectively, then this Calculation Example 2 corresponds to the case where solute permeation coefficient P2 (i.e., TOC permeation coefficient B2) cannot be used as it is as solute permeation coefficient P1 (i.e., TOC permeation coefficient B1). In such cases, the solute permeation coefficient for each reverse osmosis membrane should be measured for a mock substance, which is a known TOC component, in advance. Isopropyl alcohol can be used as the mock substance, for example. Sample water containing, for example, 100 ppb-C of isopropyl alcohol as TOC concentration is passed through the respective reverse osmosis membranes under similar conditions of, for example, a flux of 0.6 m/d and a recovery rate of 15%, and the IPA concentration of the RO permeated water is measured. Then, for respective reverse osmosis membranes, solute permeation coefficients P1 and P2 are calculated using the same procedure as described in Calculation Example 1. Since conversion factor c described in the second embodiment is expressed as c=P1/P2, the predicted TOC concentration in the RO permeated water of reverse osmosis membrane device 52 of pure water production system 50 can be obtained by performing calculation using P1=c·P2.

### Calculation Example 3

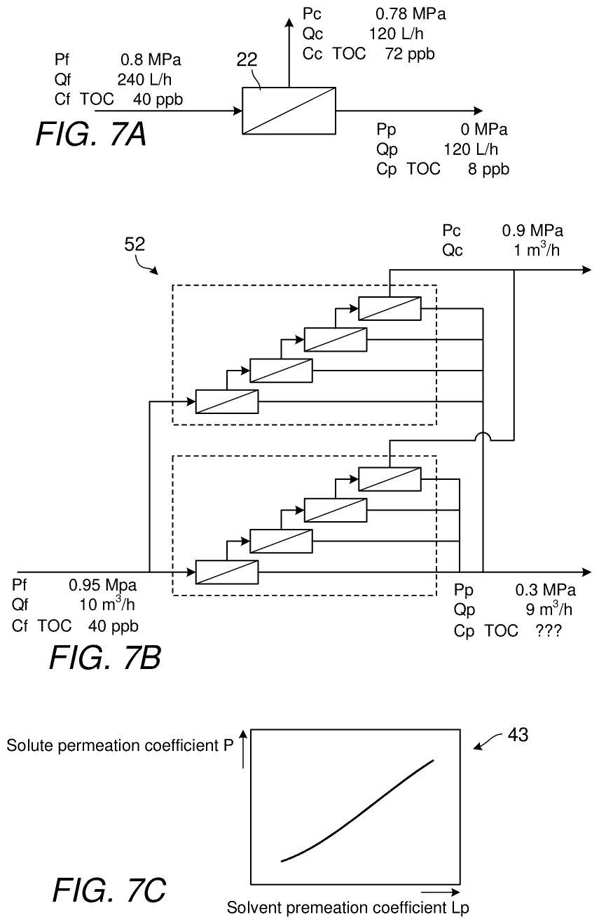

Calculation Example 3 corresponding to the third embodiment is described using FIGS. 7A to 7C. FIG. 7A shows the pressure, flow rate, and concentration in reverse osmosis membrane device 22 of system 20 for evaluation, FIG. 7B shows the configuration, pressure, flow rate, and concentration in reverse osmosis membrane device 52 of pure water production system 50, and FIG. 7C shows an example of the correlation between the solvent permeation coefficient and solute permeation coefficients. Calculation Example 3 describes calculations for estimating the TOC concentration of the RO permeated water when the membrane type of the reverse osmosis membrane in reverse osmosis membrane device 52 of pure water production system 50 is unknown. The same system 20 for system as that in Calculation Example 1 was used, and operated under the same operating conditions. Therefore, solute permeation coefficient P (i.e., TOC permeation coefficient B2) in system 20 for evaluation is 5.32×10⁻⁷ m/d. As shown in FIG. 7A, supply pressure Pf to reverse osmosis membrane device 22 in system 20 for evaluation was 0.8 MPa, pressure Pc at the concentrated water outlet was 0.78 MPa, and the pressure at the permeated water outlet was 0 MPa. If the pressure difference is represented by ΔP and the osmotic pressure for solute concentration C is represented by π(C), the following stands as described in Patent Literature 3.

$$Jv = Lp [ Δ P - π ( Cm ) - π ( Cp ) ] . \qquad ( 4 )$$

In dilute systems, the calculation may be performed by treating Cp=0. Equation (4) is used to calculate solvent permeation coefficient Lp2 (water permeation coefficient A2) in system 20 for evaluation.

On the other hand, reverse osmosis membrane device 52 in pure water production system 50 is equipped with eight membrane elements, each of which is an 8-inch element. In this reverse osmosis membrane device 52, as shown in FIG. 7B, two tracks each having four membrane elements connected in a cascade are installed in parallel. It is known that the membrane area is 37 m², but the type or model of the membrane element is unknown. Reverse osmosis membrane device 52 was operated with a recovery rate of 90% and flux Jv of 0.72 m/d. At this time, the supplied water flow rate Qf of reverse osmosis membrane device 52 was 10 m³/h and feed pressure Pf was 0.95 MPa, the concentrated water flow rate Qc was 1 m³/h and its pressure Pc was 0.9 MPa, and the permeated water flow rate Qp was 9 m³/h and its pressure Pp was 0.3 MPa. Solute concentration (TOC concentration) Cf in the supplied water was 40 ppb, the same as in system 20 for evaluation. For pure water production system 50, solvent permeation coefficient Lp1 (water permeation coefficient A1) is also calculated using Equation (4).

The solute permeation coefficient (TOC permeation coefficient) must be known to calculate the TOC concentration of the RO permeated water in pure water production system 50. In this Calculation Example, the solute permeation coefficient is also unknown because the membrane type is unknown. Therefore, the correlation of solute permeation coefficient P (TOC permeation coefficient) to solvent permeation coefficient Lp (water permeation coefficient) should be determined in advance. Solute permeation coefficient P1 (TOC permeation coefficient B1) in pure water production system 50 is obtained by applying solvent permeation coefficient Lp1 of pure water production system 50 to this correlation. The predicted value of the TOC concentration in the RO permeated water of reverse osmosis membrane device 52 is then obtained by performing the same calculations as in Calculation Example 1, etc. The correlation between solvent permeation coefficient Lp and solute permeation coefficient P can be determined by passing sample water containing the mock substance through various types of reverse osmosis membranes and calculating solvent permeation coefficient Lp and solute permeation coefficient P at that time. Specifically, isopropyl alcohol is used as the mock substance, for example, and sample water containing 100 ppb-C of isopropyl alcohol as TOC concentration, for example, is passed through the respective reverse osmosis membranes with similar conditions of, for example, a flux of 0.6 m/d and a recovery rate of 15%. FIG. 7C shows correlation diagram 43 plotting the calculated solvent permeation coefficient Lp versus solute permeation coefficient P. This correlation diagram 43 shows the correlation between solvent permeation coefficient Lp and solute permeation coefficient P. The relationship between solvent permeation coefficient Lp and solute permeation coefficient P is not necessarily expressed as a straight line, but the smaller the solvent permeation coefficient Lp is, the smaller the solute permeation coefficient P is.

### Calculation Example 4

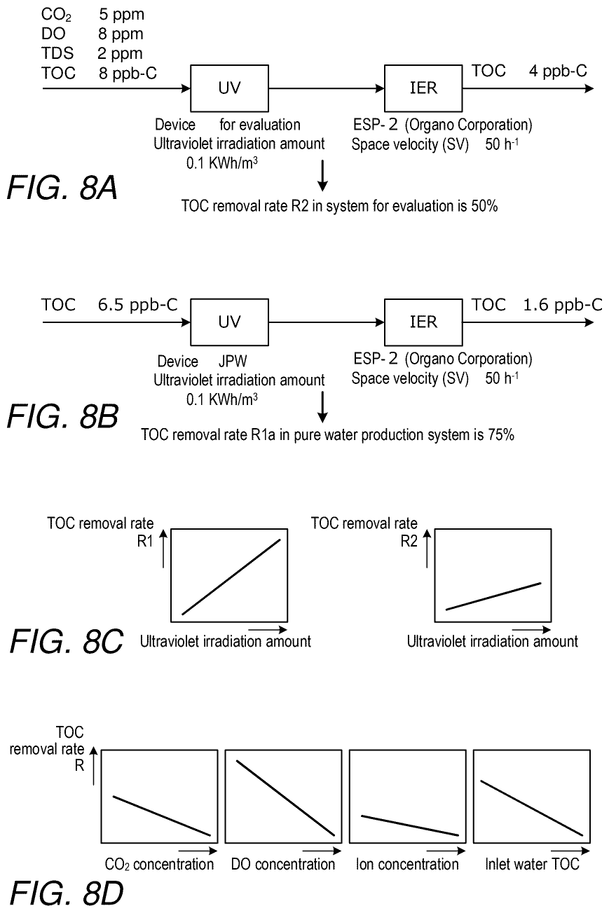

Calculation Example 4 corresponding to the fourth embodiment is described using FIGS. 8A to 8D. FIG. 8A shows ultraviolet irradiation device 23 and ion exchange device 24 of system 20 for evaluation, FIG. 8B shows ultraviolet irradiation device 53 and ion exchange device 54 of pure water production system 50, FIG. 8C shows the relationship between the amount of ultraviolet (UV) irradiation and the TOC removal rate, and FIG. 8D shows the effect of the dissolved carbon dioxide (CO₂) concentration, dissolved oxygen (DO) concentration, ion concentration, and TOC concentration on the TOC removal rate. Ultraviolet irradiation device 23 of system 20 for evaluation is an ultraviolet irradiation device used for evaluation. The amount of the ultraviolet irradiation was 0.1 kWh/m³. A cartridge polisher ESP-2, manufactured by Organo Corporation, was used as ion exchange device 24, and its space velocity (SV) of the water flow was 50 h⁻¹. The dissolved carbon dioxide (CO₂) concentration of the inlet water of UV irradiation unit 23, i.e., the RO permeated water from reverse osmosis membrane device 22 at the preceding stage, was 5 ppm, the dissolved oxygen (DO) concentration was 8 ppm, the total dissolved solids (TDS) was 2 ppm, and the TOC concentration was 8 ppb-C. The TOC concentration in the treated water (pure water) of ion exchange device 24 was 4.0 ppb-C, and TOC removal rate R was calculated to be 50%. The TOC concentration here relates to unknown TOC components derived from the raw water.

As ultraviolet irradiation device 53 of pure water production system 50, a JPW manufactured by Photoscience Japan Corporation was used with amount of ultraviolet irradiation of 0.1 kWh/m³. As ion exchange device 54, a cartridge polisher ESP-2, manufactured by Organo Corporation was used, and its space velocity (SV) of the water flow was 50 h⁻¹. It is assumed here that the RO permeated water discharged from reverse osmosis membrane device 52 in Calculation Example 1 is supplied to ultraviolet irradiation device 53, the TOC concentration value (predicted value) at the inlet water of ultraviolet irradiation device 53 is 6.5 ppb-C. The goal of Calculation Example 4 is to predict the TOC concentration in the treated water (pure water) of ion exchange device 54 in pure water production system 50 with respect to unknown TOC components derived from the raw water.

In the example shown here, system 20 for evaluation and pure water production system 50 have the same ultraviolet irradiation amount, but differs in the model of the ultraviolet irradiation devices, which results in different TOC removal efficiencies relative to the ultraviolet irradiation amount. Thus, removal rate R2 of 50% calculated for system 20 for evaluation cannot be directly applied to pure water production system 50. Therefore, the TOC removal rate is corrected based on differences in ultraviolet irradiation devices. The relationship between the ultraviolet irradiation amount and the TOC removal rate is determined in advance by passing sample water containing a mock substance, which is a TOC component, through ultraviolet irradiation devices 23, 53 of system 20 for evaluation and pure water production system 50, respectively, to conduct ultraviolet oxidation treatment of the sample water while changing the ultraviolet irradiation amount. For example, the ultraviolet oxidation treatment is performed on the sample water containing 10 ppb-C of isopropyl alcohol as a mock substance, while varying the ultraviolet irradiation amount in the range of 0.1 to 1 kWh/m³. FIG. 8C shows the relationship between the ultraviolet irradiation amount and the TOC removal rate thus obtained. The graph of TOC removal rate R1 was obtained for the pure water production system 50, and the graph of TOC removal rate R2 was obtained for system 20 for evaluation. The ratio (R1/R2) of TOC removal rates R1 and R2 for the mock substance obtained in this way can be used as a correction factor to determine TOC removal rate R1a for unknown TOC components originating from the raw water by multiplying TOC removal rate R2 obtained previously for system 20 for evaluation by this correction factor. In the example shown here, TOC removal rate R1a is 75%, and the TOC concentration in the treated water of pure water production system 50 is expected to be 1.6 ppb.

Although we have described an example in which the TOC removal rate is corrected for differences in the configuration or the like of the ultraviolet irradiation devices, it is known that the TOC removal rate in the ultraviolet oxidation-ion exchange treatment is also affected by the concentration of each component in the raw water, such as the concentrations of dissolved carbon dioxide (CO₂), dissolved oxygen (DO), ionic impurities, and TOC. Therefore, in the same way that the TOC removal rate is corrected based on the configuration of the ultraviolet irradiation device, it is preferable to determine, for each concentration item, the relationship between the concentration and TOC removal rate R in advance using a mock substance while keeping the ultraviolet irradiation amount, and correct the TOC removal rate to be used for pure water production system 50 based on the relationship thus determined. FIG. 8D shows an example of the relationship thus obtained between concentration of each concentration item and TOC removal rate.

The correction of the TOC removal rate by dissolved carbon dioxide concentration is described as an example. It is supposed that the concentration of dissolved carbon dioxide in pure water production system 50 is 1 ppm and that in system 20 for evaluation system 20 is 10 ppm. From the graph of the dissolved carbon dioxide concentration versus TOC removal rate (which is for the mock substance) shown in FIG. 8D, TOC removal rate Ra for 1 ppm and TOC removal rate RB for 10 ppm are obtained, and Ra/RB is used as the correction factor for the dissolved carbon dioxide concentration. This correction factor is further multiplied to TOC removal rate R1a obtained after the correction for the ultraviolet irradiation device is made to obtain TOC removal rate R1b. TOC removal rate R1b can be used to predict the TOC concentration in the treated water of pure water production system 50, taking into account differences in the dissolved carbon dioxide concentration. Although the correction factor is calculated here based only on the dissolved carbon dioxide concentration, one or more of several concentration items, such as the dissolved carbon dioxide concentration, dissolved oxygen concentration, ionic impurity concentration, TOC concentration, etc. can be used to calculate the correction factor.

### REFERENCE SIGNS LIST

10 Water quality prediction system;
20 System for evaluation;
22, 52 Reverse osmosis membrane device (RO);
23, 53 Ultraviolet irradiation device (UV);
24, 54 Ion exchange device (IER);
25 Measurement instrument;
26 Evaluation calculation unit;
50 Pure water production system; and
51 Raw water tank

1. A water quality prediction system for predicting quality of treated water in a water treatment system equipped with a first water treatment device that performs a unit operation on water to be treated when the water to be treated is supplied to the water treatment system and the water treatment system is operated based on first operating parameters, the water quality prediction system comprising:
a system for evaluation that is equipped with a second water treatment that performs same unit operation as the first water treatment device, that is supplied with the water to be treated which is to be supplied to the water treatment system, and that is operated according to second operating parameters; and
a calculation unit that calculate a predicted value of solute concentration in the treated water of the water treatment system based on quality of the water to be treated, quality of treated water in the system for evaluation, the first operating parameters, and the second operating parameters.

2. The water quality prediction system according to claim 1, wherein
the first water treatment device comprises a first reverse osmosis membrane device equipped with a first reverse osmosis membrane whose solute permeation coefficient is a first solute permeation coefficient,
the second water treatment device comprises a second reverse osmosis membrane device equipped with a second reverse osmosis membrane whose solute permeation coefficient is a second solute permeation coefficient, and
the calculation unit is configured to
obtain the second solute permeation coefficient on the quality of the water to be treated, quality of permeated water of the second reverse osmosis membrane, and the second operating parameters,
estimate the first solute permeation coefficient based on the solute permeation coefficient of the second reverse osmosis membrane, and
calculate the predicted value of solute concentration on permeated water of the first reverse osmosis membrane from the first solute permeation coefficient, the quality of the water to be treated, and the first operating parameters.

3. The water quality prediction system according to claim 2, wherein the calculation unit is configured to multiply the second solute permeation coefficient by a conversion factor based on a combination of a membrane type of the first reverse osmosis membrane and a membrane type of the second reverse osmosis membrane to obtain the first solute permeation coefficient.

4. The water quality prediction system according to claim 2, wherein the calculation unit is configured to
obtain a first water permeation coefficient, which is a water permeation coefficient of the first reverse osmosis membrane, and a second water permeation coefficient, which is water permeation coefficient of the second reverse osmosis membrane, and
use the first water permeation coefficient, the second water permeation coefficient, and the second solute permeation coefficient to estimate the first solute permeation coefficient, based on correlation between a ratio of the first water permeation coefficient to the second water permeation coefficient and a ratio of the first solute permeation coefficient to the second solute permeation coefficient.

5. The water quality prediction system according to claim 2, wherein
the water treatment system further comprises: a first ultraviolet irradiation device provided at a subsequent stage of the first reverse osmosis membrane; and a first ion exchange device provided at a subsequent stage of the first ultraviolet irradiation device,
the system for evaluation further comprises: a second ultraviolet irradiation device provided at a subsequent stage of the second reverse osmosis membrane; and a second ion exchange device provided at a subsequent stage of the second ultraviolet irradiation device, and
the calculation unit is configured to
obtain a solute removal rate of attained by both the second ultraviolet irradiation device and the second ion exchange device,
correct the solute removal rate based on the first operating parameters and the second operating parameters, and
use the corrected solute removal rate to calculate a predicted value of solute concentration of treated water of the first ion exchange device.

6. The water quality prediction system according to claim 5, wherein the calculation unit is configured to use the predicted value of solute concentration of the permeated water of the first reverse osmosis membrane in addition to the corrected solute removal rates to calculate the predicted value of solute concentration of the treated water of the first ion exchange device.

7. The water quality prediction system according to claim 1, wherein
the first water treatment device comprises a first ultraviolet irradiation device and a first ion exchange device provided at a subsequent stage of the first ultraviolet irradiation device,
the second water treatment device comprises a second ultraviolet irradiation device and a second ion exchange device provided at a subsequent stage of the second ultraviolet irradiation device, and
the calculation unit is configured to
obtain a solute removal rate of attained by both the second ultraviolet irradiation device and the second ion exchange device,
correct the solute removal rate based on the first operating parameters and the second operating parameters, and
calculates a predicted value of solute concentration of treated water of the first ion exchange device from the corrected solute removal rate and the quality of the water to be treated.

8. The water quality prediction system according to claim 5, wherein the calculation unit is configured to correct the solute removal rate based on relationship between ultraviolet irradiation amount and the solute removal rate, the relationship being obtained by passing a sample water containing a mock substance which is a known solute component through the first ultraviolet irradiation device and the second ultraviolet irradiation device.

9. A water quality prediction method for predicting quality of treated water in a water treatment system equipped with a first water treatment device that performs a unit operation on water to be treated water when the water to be treated is supplied to the water treatment system and the water treatment system is operated based on first operating parameters, comprising:
supplying the water to be treated, which is to be supplied to the water treatment system, to a system for evaluation to operate the system for evaluation based on second operating parameters, the system for evaluation being equipped with a second water treatment device that performs same unit operation as the first water treatment device; and
calculating a predicted value of solute concentration of the treated water in the water treatment system based on quality of the water to be treated, quality of treated water in the system for evaluation, the first operating parameters, and the second operating parameters.

10. The water quality prediction system according to claim 7, wherein the calculation unit is configured to correct the solute removal rate based on relationship between ultraviolet irradiation amount and the solute removal rate, the relationship being obtained by passing a sample water containing a mock substance which is a known solute component through the first ultraviolet irradiation device and the second ultraviolet irradiation device.
